(19) United States
(12) Patent Application Publication
(10) Pub. No.: US 2013/0323193 A1
(43) Pub. Date: Dec. 5, 2013

(54) OIL-SOLUBLE DEODORANT COMPOSITION AND METHOD FOR DEODORIZING AMMONIA ODOR AND AMINE ODOR

(71) Applicants: Takashi Kawano, Tokyo (JP); Takahiro Ishikawa, Kanagawa (JP); Sadahiko Yamazaki, Tokyo (JP); Kunihide Hoshino, Kanagawa (JP)

(72) Inventors: Takashi Kawano, Tokyo (JP); Takahiro Ishikawa, Kanagawa (JP); Sadahiko Yamazaki, Tokyo (JP); Kunihide Hoshino, Kanagawa (JP)

(73) Assignee: TAKASAGO INTERNATIONAL CORPORATION, Tokyo (JP)

(21) Appl. No.: 13/985,762

(22) PCT Filed: Mar. 2, 2012
(86) PCT No.: PCT/JP2012/056044

§ 371 Date: Aug. 15, 2013

(30) Foreign Application Priority Data
Mar. 4, 2011 (JP) 2011-048207

(51) Int. Cl. A61L 9/01

(52) U.S. Cl. CPC A61L 9/01
USPC 424/76.1; 562/590; 562/595

(57) ABSTRACT

The present invention provides an oil-soluble deodorant composition that is highly safe, efficienctly eliminates odors of ammonia, amines and the like and is free of an irritant odor and the like. The present invention relates to an oil-soluble deodorant composition containing at least one of a dimer acids and a trimer acids as an active ingredient.

### TECHNICAL FIELD

The present invention relates to an oil-soluble deodorant composition that can effectively eliminate odors of ammonia, amines and the like.

### BACKGROUND ART

As conventional oil-soluble deodorants used for elimination of odors of amines, metacrylic acid esters (Patent Literature 1 and Patent Literature 2), aldehydes of fragrance compositions (Patent Literature 3) are reported.

In addition, examples of a substance having a dicarboxylic acid structure include succinic acid, phthalic acid, dimethyl eicosadienoic acid, tetradecanedioic acid and the like. Since the substance cannot be dissolved in an oil-soluble solvent such as hydrocarbon solvent, only aqueous deodorants including such a substance are inevitably developed.

### CITATION LIST

### Patent Literature

[PLT 1] JP-A-48-44441
[PLT 2] JP-A-1-129854
[PLT 3] JP-A-2001-303090

### SUMMARY OF INVENTION

### Technical Problem

However, lauryl methacrylate generally used as methacrylic acid esters has a disadvantage of low deodorization effect on odor of amines. In addition, aldehydes and the like have an effect of deodorizing odor of amines, but have a disadvantage of limited use, since the substances exhibiting deodorization effects impart unpleasant feeling to users due to specific strong irritant odors.

Accordingly, an object of the present invention is to provide an oil-soluble deodorant composition that is highly safe, can efficiently eliminate malodors of ammonia, amines and the like, and is free of an irritant odor and the like.

### Solution to Problem

As a result of intensive studies to solve the above-mentioned problems, the present inventors have found that dimer acids and trimer acids exhibit excellent deodorization effects on odors of ammonia, amines such as trimethylamine and the like. The present invention has been completed based on this finding.

That is, the present invention provides the following oil-soluble deodorant composition and method for deodorizing ammonia odor or amine odor.

(1) An oil-soluble deodorant composition comprising at least one of a dimer acids and a trimer acids as an active ingredient.

(2) The oil-soluble deodorant composition according to (1), wherein the dimer acids is at least one of a dimer acid and a hydrogenated dimer acid.

(3) The oil-soluble deodorant composition according to (1) or (2), wherein the trimer acids is at least one of a trimer acid and a hydrogenated trimer acid.

(4) The oil-soluble deodorant composition according to any one of (1) to (3), further comprising at least one of a fatty acid metal salt and an organic acid metal salt.

(5) The oil-soluble deodorant composition according to (4), wherein the metal of the at least one of the fatty acid metal salt and the organic acid metal salt is one or more selected from the group consisting of sodium, potassium, calcium, copper, iron, zinc and magnesium.

(6) The oil-soluble deodorant composition according to (4) or (5), wherein the fatty acid metal salt is zinc ricinoleate.

(7) The oil-soluble deodorant composition according to any one of (1) to (6), wherein a total content of the dimer acids and trimer acids is 0.1% by weight or more.

(8) A method for deodorizing ammonia odor or amine odor using at least one of a dimer acids and a trimer acids.

(9) A method for deodorizing ammonia odor or amine odor, comprising applying at least one of a dimer acids and a trimer acids to a subject to be deodorized, or applying at least one of a dimer acids and a trimer acids near a subject to be deodorized.

### Advantageous Effects of Invention

The oil-soluble deodorant composition of the present invention contains at least one of dimer acids and trimer acids as an active ingredient, and thus the composition exhibits excellent deodorization effects on odors of ammonia, amines such as trimethylamine and the like, as compared to conventional oil-soluble deodorant compositions.

In addition, the dimer acids contained in the oil-soluble deodorant composition of the present invention have a skeleton of dicarboxylic acid and the trimer acids therein have a skeleton of tricarboxylic acid. The dimer and trimer acids are readily dissolved in oil-soluble solvent such as hydrocarbon and are thus considerably useful.

Furthermore, dimer acids and trimer acids are liquid substances that are flowable in spite of their large molecular weights and the oil-soluble deodorant composition of the present invention is excellent in terms of handling.

In addition, since the oil-soluble deodorant composition of the present invention is free of an irritant odor or the like, it is highly safe and may be added to various products, thus being considerably useful.

### DESCRIPTION OF EMBODIMENTS

Hereinafter, the present invention will be described in detail. In the present invention, “% by weight” has the same meaning as “% by mass”.

The oil-soluble deodorant composition of the present invention contains at least one of dimer acids and trimer acids as an active ingredient.

In the present invention, dimer acids refer to dicarboxylic acid composed of a dimer of unsaturated fatty acid, and a hydrogenated dimer acid obtained by reducing an unsaturated bond commonly contained in the dimer acid. The dimer acids are preferably at least one of the dimer acid and the hydrogenated dimer acid. The dimer acid and hydrogenated dimer acid may be used alone or in combination thereof.

In the present invention, the dimer acid is dicarboxylic acid composed of a dimer of unsaturated fatty acid. The unsaturated fatty acid constituting the dimer acid preferably has 10 or more carbon atoms and 24 or less carbon atoms, more preferably 18 carbon atoms.

Examples of the unsaturated fatty acid having 18 carbon atoms include oleic acid, linoleic acid, linolenic acid and the like. The dimer acid can be prepared by dimerizing these unsaturated fatty acids.

Generally available dimer acid is obtained by purifying plant-derived substances such as rapeseed oil, soybean oil and tall oil, and separating the dimer from low polymers of unsaturated fatty acids.

Examples of the dimer acid include aliphatic dimer acid, cyclic dimer acid such as alicyclic dimer acid, aromatic dimer acid, dicyclic dimer acid and the like. The dimer acid may be used singly or in combination of two or more types thereof.

The hydrogenated dimer acid refers to a dimer acid obtained by reducing an unsaturated bond commonly contained in the dimer acid through addition of hydrogen to the dimer acid. The hydrogenated dimer acid used for the present invention may be obtained by reducing respective aforementioned dimer acid through addition of hydrogen.

In particular, preferable examples thereof include hydrogenated dimmer acid obtained by reducing a dimer acid in which the number of carbon atoms of unsaturated fatty acid constituting the dimer acid is 18 through addition of hydrogen, and more preferable examples thereof include hydrogenated dimmer acid obtained by reducing a dimer acid in which unsaturated fatty acid constituting the dimer acid is oleic acid, linoleic acid or linolenic acid through addition of hydrogen.

The hydrogenated dimer acid may be used singly or in combination of two or more types thereof.

In the present invention, trimer acids refers to tricarboxylic acid composed of a trimer of unsaturated fatty acid, and a hydrogenated trimer acid obtained by reducing an unsaturated bond commonly contained in the trimer acid. The trimer acids are preferably at least one of the trimer acid and the hydrogenated trimer acid. The trimer acid and hydrogenated trimer acid may be used alone or in combination thereof.

In the present invention, the trimer acid is tricarboxylic acid composed of a trimer of unsaturated fatty acid. The unsaturated fatty acid constituting the trimer acid preferably has 10 or more carbon atoms and 24 or less carbon atoms, more preferably 18 carbon atoms.

Examples of the unsaturated fatty acid having 18 carbon atoms include oleic acid, linoleic acid, linolenic acid and the like. The trimer acid can be prepared by trimerizing these unsaturated fatty acids.

Generally available trimer acid is obtained by purifying plant-derived substances such as rapeseed oil, soybean oil and tall oil, and separating trimers from low polymers of unsaturated fatty acid.

Examples of the trimer acid include aliphatic trimer acid, cyclic trimer acid such as alicyclic trimer acid, aromatic trimer acid, dicyclic trimer acid and the like. The trimer acid may be used singly or in combination of two or more types thereof.

The hydrogenated trimer acid refers to a trimer acid obtained by reducing an unsaturated bond commonly contained in the trimer acid through addition of hydrogen to the trimer acid. The hydrogenated trimer acid used for the present invention may be obtained by reducing respective aforementioned trimer acids through addition of hydrogen.

In particular, preferable examples thereof include hydrogenated trimer acid obtained by reducing a trimer acid in which the number of carbon atoms of unsaturated fatty acid constituting the trimer acid is 18 through addition of hydrogen, and more preferable examples thereof include hydrogenated trimer acid obtained by reducing a trimer acid in which the unsaturated fatty acid constituting the trimer acid is oleic acid, linoleic acid or linolenic acid through addition of hydrogen.

The hydrogenated trimer acid may be used singly or in combination of two or more types thereof.

The dimer acids and trimer acids used for the present invention are preferably dimer acid, hydrogenated dimer acid and hydrogenated trimer acid in terms of preferred properties such as pale color tone and substantial colorlessness of a deodorization composition. Of these, hydrogenated dimer acid is more preferred.

In particular, hydrogenated dimer acid is colorless and odorless and dimer acid to which hydrogen is not added is slightly yellow in color. Accordingly, hydrogenated dimer acid is most preferably in terms of color tone and odor tone.

The dimer acids and trimer acids used for the present invention may be commercially available products. Examples of commercially available products include “PRIPOL1017”, “PRIPOL1022”, and “PRIPOL1029” (all of them have a mix ratio of 80% by weight of dimer acid and 20% by weight of trimer acid); “PRIPOL1012” and “PRIPOL1013” (all of them have 95% by weight of dimer acid); “PRIPOL1009” (99% by weight of hydrogenated dimer acid); “PRIPOL1025” (has a mix ratio of 80% by weight of hydrogenated dimer acid and 20% by weight of hydrogenated trimer acid) (all of them are manufactured by CRODA Inc.) and the like.

In the oil-soluble deodorant composition of the present invention, a total content of the dimer acids and the trimer acids contained as an active ingredient is strictly not limited, and depends on the concentration of ammonia and amine to be deodorized, and is 0.1% by weight or more, more preferably 0.1 to 100% by weight, even more preferably 1 to 80% by weight, based on the total weight of the oil-soluble deodorant composition.

When the total content of the dimer acids and the trimer acids in the oil-soluble deodorant composition is 0.1% by weight or more, the deodorization effect on ammonia and amines can be improved.

A mix ratio of the dimer acids and the trimer acids contained as an active ingredient is strictly not limited and depends on use form, and a mix ratio (weight ratio) of the dimer acids to the trimer acids (dimmer acids: trimer acids) is preferably 0:10 to 10:0, more preferably 1:9 to 9:1, even more preferably 2:1 to 4:1.

Preferably, the oil-soluble deodorant composition of the present invention further contains at least one of a fatty acid metal salt and an organic acid metal salt. The deodorant composition of the present invention exhibits superior deodorization effects on malodors of hydrogen sulfate, mercaptan and the like as well as ammonia and amines by incorporating the at least one of a fatty acid metal salt and an organic acid metal salt.

Examples of the fatty acid of the fatty acid metal salt include ricinoleic acid, stearic acid, myristic acid, undecylenic acid, lauric acid and the like. Of these, ricinoleic acid is most preferable in terms of solubility and safety.

Examples of the organic acid of organic acid metal salt include gluconic acid, citric acid, tartaric acid, oxo acid and the like. Of these, gluconic acid is preferable in terms of solubility.

Examples of the metal of metal salt of fatty acid metal salt and organic acid metal salt include sodium, potassium, calcium, copper, iron, zinc, magnesium and the like. Of these, zinc is particularly preferable in terms of deodorization effects.

At least one of fatty acid metal salt and organic acid metal salt may be one selected therefrom or a combination of two or more types thereof.

In the present invention, among the fatty acid metal salt and the organic acid metal salt, the fatty acid metal salt is preferable in terms of solubility in oils, and fatty acid zinc salt is more preferable. Among the fatty acid zinc salts, zinc ricinoleate is most preferable in terms of solubility and safety.

In the oil-soluble deodorant composition of the present invention, a total content of the fatty acid metal salt and the organic acid metal salt is not strictly limited, and depends on the concentration of hydrogen sulfate and mercaptans, and is preferably 0.0001% by weight or more, more preferably 0.001 to 50.0% by weight, even more preferably 0.01 to 20% by weigh, based on the total weight of the oil-soluble deodorant composition.

When the total content of fatty acid metal salt and organic acid metal salt in the oil-soluble deodorant composition of the present invention is 0.0001% by weight or more, hydrogen sulfate and mercaptans can be deodorized.

The oil-soluble deodorant composition of the present invention contains at least one of the dimer acids and the trimer acids and may be further combined with other deodorant. In addition, the oil-soluble deodorant composition of the present invention may further contain solvents such as oils and fats, waxes, hydrocarbons, alcohols, glycols, glycol ethers, glycol esters, ethers, esters, ketones and fatty acids, and common additives such as antioxidants, light stabilizers, pH controllers, preservatives, fragrances, surfactants, pigments, UV absorbers, antibacterial agents, gelling agents and thickening agents.

Examples of oils and fats include avocado oil, almond oil, olive oil, hardened oil, coconut oil, castor oil, hardened castor oil, soybean oil, sesame oil and the like. The amount of oils and fats added to the oil-soluble deodorant composition of the present invention is not strictly limited and is preferably 0.1% by weight or more.

Examples of the waxes include candelilla wax, jojoba oil, bees wax and the like. The amount of waxes added to the oil-soluble deodorant composition of the present is not strictly limited and is preferably 0.1% by weight or more.

Examples of the hydrocarbons include normal paraffin, isoparaffin, paraffin, squalene, liquid paraffin, vaseline, toluene, xylene, mineral spirit, isohexane, normal decane, normal heptane and the like. The amount of hydrocarbons added to the oil-soluble deodorant composition of the present invention is not strictly limited and is preferably 0.1% by weight or more.

Examples of the alcohols include ethyl alcohol, isopropyl alcohol, butanol, isopropanol, isostearyl alcohol, 1,3-butanediol and the like. The amount of alcohols added to the oil-soluble deodorant composition of the present invention is not strictly limited and is preferably 0.1% by weight or more.

Examples of the glycols include propylene glycol, dipropylene glycol, hexylene glycol, butylene glycol, glycerin and the like. The amount of glycols added to the oil-soluble deodorant composition of the present invention is not strictly limited and is preferably 0.1% by weight or more.

Examples of the glycol ethers include propylene glycol monomethyl ether, dipropylene glycol monomethyl ether, dipropylene glycol dimonomethyl ether, 3-methoxy-3-methyl-1-butanol and the like. The amount of glycol ethers added to the oil-soluble deodorant composition of the present invention is not strictly limited and is preferably 0.1% by weight or more.

Examples of the glycol esters include propylene glycol methyl ether acetate, propylene glycol diacetate, dipropylene glycol methyl ether acetate and the like. The amount of glycol esters added to the oil-soluble deodorant composition of the present invention is not strictly limited and is preferably 0.1% by weight or more.

Examples of the ethers include butyl carbitol, 2-methoxy ethanol and the like. The amount of esters added to the oil-soluble deodorant composition of the present invention is preferably 0.1% by weight or more.

Examples of the esters include benzyl benzoate, triethyl citrate, decyl oleate, diethyl phthalate and the like. The amount of esters added to the oil-soluble deodorant composition of the present invention is preferably 0.1% by weight or more.

Examples of the ketones include acetone, diacetone alcohol, cyclohexanone and the like. The amount of ketones added to the oil-soluble deodorant composition of the present invention is 0.1% by weight or more.

Examples of the fatty acids include isostearic acid, undecylenic acid, oleic acid, stearic acid, palmitic acid, myristic acid, coconut oil fatty acid and the like. The amount of ketones added to the oil-soluble deodorant composition of the present invention is preferably 0.1% by weight or more.

Examples of the surfactants include cationic surfactants, anionic surfactants, nonionic surfactants, amphiprotic surfactants and the like. The amount of surfactant added to the oil-soluble deodorant composition of the present invention is not strictly limited and is preferably 0.1% by weight or more in terms of solubilization of fragrances.

Examples of fragrances include natural essential oils such as lemon oil, orange oil, lime oil, bergamot oil, lavandin oil, lavender oil, geranium oil and rose oil, sandalwood oil, hydrocarbons such as α-pinene, β-pinene, limonene and p-cymene, and aliphatic alcohols such as octanol and p-tert-butylcyclohexanol, terpene alcohols such as menthol, citronellol and geraniol, aromatic alcohols such as benzyl alcohol and phenylethyl alcohol, aliphatic aldehydes, terpene aldehydes, aromatic aldehydes, acetals, chain ketones, cyclic ketones such as damascone, β-ionone and methyl ionone, terpene ketones such as carvone, menthone, isomenthone and camphor, aromatic ketones such as acetophenone and raspberry ketone, ethers such as dibenzyl ether, oxides such as linalool oxide and rose oxide, musks such as cyclopentadecanolide and cyclohexadecanolide, lactones such as γ-nonalactone, γ-undecalactone and coumarin, aliphatic esters such as ester acetate and ester propionate, aromatic esters such as ester benzoate and ester phenylacetate and the like. The amount of fragrance added to the oil-soluble deodorant composition of the present invention is preferably 0.001% by weight or more.

The oil-soluble deodorant composition of the present invention may be combined with a known carrier. Examples of the carrier include solvents such as alcohols, glycols, oils and fats, waxes, hydrocarbons, glycol ethers, glycol esters, ethers, esters and ketones, and liquid carriers such as fragrance compositions, gas carriers such as LPG, and solid carriers such as calcium silicate, silica gel, gelling agents and thickening agents.

The content of carrier in the oil-soluble deodorant composition of the present invention is strictly not limited, and depends on the concentration of malodor, and is preferably 0.001 to 50% by weight, more preferably 0.001 to 30% by weight.

In addition, the form of the oil-soluble deodorant composition may be a liquid form, a powder form, a gel form, a granular form and aerosol form, depending on application.

The deodorant composition of the present invention is oily, but may be mixed with an aqueous component by combining surfactants, a solvent such as alcohols, glycols, glycol ethers, glycol esters, ethers, esters, ketones and fatty acid, a fragrance composition, or the like.

By mixing the components, the deodorant composition may be used for any of oil-soluble final products and water-soluble final products.

The oil-soluble deodorant composition of the present invention may be used directly as a final product such as deodorant, air freshner and deodorizer. That is, the oil-soluble deodorant composition of the present invention may be used as an oil-soluble composition for deodorants, an oil-soluble composition for air freshners, an oil-soluble composition for deodorizers or the like.

The oil-soluble deodorant composition of the present invention may be added to final products such as household products and toiletry products.

Examples of the household products include deodorizing spray, detergents for clothes, bleaching agents for clothes, conditioners for clothes, detergents for dishes, detergents for bathrooms, detergents for toilets and the like.

The content of the oil-soluble deodorant composition of the present invention in the household products is not strictly limited, and depends on the concentration of malodor, and is preferably 0.001 to 30% by weight, more preferably 0.001 to 10% by weight, based on the total weight of the household product.

Examples of the toiletry products include body detergents, deodorants, shampoos, rinses, conditioners, treatments, hair packs, bleaching agents, permernant agents, hair color agents and the like.

The content of the oil-soluble deodorant composition of the present invention in the toiletry product is not strictly limited, and depends on the concentration of malodor, and is preferably 0.001 to 30% by weight, more preferably 0.001 to 10% by weight, based on the total weight of the toiletry product.

The oil-soluble deodorant composition of the present invention may be used for deodorizing or odor-eliminating food wastes, clothes, refrigerators, dressers/closets/lockers, indoors, vehicles, toilets, bathrooms, pet products, plants, industrial waste liquids, air cleaner, air conditioners, deodorizing devices, filters for air blower and ventilator, sewage disposal plants, cattle stalls, and dust and garbage disposal plants.

The oil-soluble deodorant composition of the present invention may be directly applied to a subject to be deodorized or odor-eliminated, or applied near the subject.

The dimer acids and trimer acids contained as active ingredients in the oil-soluble deodorant composition of the present invention exhibit excellent deodorization effects on ammonia and amines such as trimethylamine. When the oil-soluble deodorant composition of the present invention is applied to toilet odors, waster odors, indoor odors or pet odors that contain a great amount of these malodor components and occur in daily life, generation of malodors can be effectively prevented due to the deodorization effects.

Furthermore, by using at least one of the dimer acids and the trimer acids in conjunction with at least one of a fatty acid metal salt and an organic acid metal salt, excellent deodorization effect on hydrogen sulfate, mercaptans and the like can be obtained.

### EXAMPLE

Hereinafter, the present invention will be described with reference to Examples and Comparative Examples in more detail and the present invention is not limited thereto.

### Example 1

### Deodorization of Effects of Hydrogenated Dimer Acid, Mixture of Hydrogenated Dimer Acid and Hydrogenated Trimer Acid, and Mixture of Dimer Acid and Trimer Acid on Ammonia Odor

To a filter paper having φ 55 mm, as a sample, 1.0 g of 20% by weight of an isoparaffin solution of each of hydrogenated dimer acid [“PRIPOL1009”(trade name)(manufactured by manufactured by CRODA Inc.)], a mixture of 80% by weight of hydrogenated dimer acid and 20% by weight of hydrogenated trimer acid [“PRIPOL1025″(trade name, manufactured by CRODA Inc.)], or a mixture of 80% by weight of dimer acid and 20% by weight of trimer acid [“PRIPOL1017”(trade name, manufactured by CRODA Inc)”] was added dropwise, followed by addition to a 5 L Tedlar bag, sealing and injection of an odorless air.

Then, an ammonia gas was injected into the Tedlar bag, the concentration of ammonia in the container after 60 minutes and 120 minutes was measured, respectively, using a gas detecting tube (manufactured by Gestec Service, Inc.) and a deodorization rate was calculated by the following equation. The results are shown in Table 1. In addition, an initial concentration of ammonia was 80 ppm.

(Equation for Calculation of Deodorization Rate)

Deodorization rate(%)=[(C−S)/C]×100

S: Concentration of odor gas in a container containing a sample (ppm)

C: Concentration of odor gas in a container as a control (ppm)

The control used herein was obtained by adding dropwise 1.0 g of isoparaffin to a filter paper.

### Comparative Example 1

### Deodorization of Effects of Lauryl Methacrylate on Ammonia Odor

Lauryl methacrylate was used as a sample and the concentration of ammonia was measured through the same adjustment as in Example 1. The results are shown in Table 1.

TABLE 1
Results of deodorization test on ammonia odor (initial concentration: 80 ppm)

| | | Deodorization | Deodorization |
|---|---|---|---|
| | | rate after 60 min | rate after 120 min |
| | Sample name | (%) | (%) |
| Example 1-1 | Hydrogenated dimer acid | 86.0 | 97.0 |
| Example 1-2 | Mixture of 80% by weight of | 83.0 | 96.0 |
| | hydrogenated dimer acid and 20% by | | |
| | weight of hydrogenated trimer acid | | |
| Example 1-3 | Mixture of 80% by weight of dimer | 83.0 | 96.0 |
| | acid and 20% by weight of trimer acid | | |
| Comparative | Lauryl methacrylate | 12.0 | 15.0 |
| Example 1 | | | |

As can be seen from Table 1, the cases of Examples 1-1 to 1-3 containing at least one of dimer acids and trimer acids exhibited superior deodorization effects on ammonia odor, as compared to the case of lauryl methacrylate.

### Example 2

### Deodorization of Effects of Hydrogenated Dimer Acid, Mixture of Hydrogenated Dimer Acid and Hydrogenated Trimer Acid, and Mixture of Dimer Acid and Trimer Acid on Trimethylamine Odor

The same adjustment as in Example 1 was performed except that trimethylamine was used as a malodor substance and the deodorization effect on trimethylamine was measured. In addition, an initial concentration of trimethylamine was adjusted to 25 ppm. The results are showin in Table 2.

### Comparative Example 2

### Deodorization of Effects of Lauryl Methacrylate on Trimethylamine Odor

Lauryl methacrylate was used as a sample and the concentration of trimethylamine was measured in the same adjustment method as in Example 2. The results are showin in Table 2.

TABLE 2
Results of deodorization test on trimethylamine odor (initial concentration 25 ppm)

| | | Deodorization | Deodorization |
|---|---|---|---|
| | | rate after 60 min | rate after 120 min |
| | Sample name | (%) | (%) |
| Example 2-1 | Hydrogenated dimer acid | 84.0 | 92.0 |
| Example 2-2 | Mixture of 80% by weight of | 80.0 | 88.0 |
| | hydrogenated dimer acid and 20% by | | |
| | weight of hydrogenated trimer acid | | |
| Example 2-3 | Mixture of 80% by weight of dimer | 80.0 | 88.0 |
| | acid and 20% by weight of trimer acid | | |
| Comparative | Lauryl methacrylate | 00.0 | 00.0 |
| Example 2 | | | |

As can be seen from Table 2, the cases of Examples 2-1 to 2-3 containing at least one of dimer acids and trimer acids exhibited superior deodorization effects on trimethylamine odor, as compared to the case of lauryl methacrylate.

### Example 3

### Deodorization of Effects of Mixture of Hydrogenated Dimer Acid and Zinc Ricinoleate on Ammonia and Hydrogen Sulfate

To a filter paper having φ55 mm, 1.0 g of 20% by weight of an isoparaffin solution of a sample in which 50% by weight of hydrogenated dimer acid, 1.0% by weight of zinc ricinoleate and 49.0% by weight of an alcohol-based solvent are mixed and adjusted, was added dropwise, followed by addition to a 5 L Tedlar bag, sealing and injection of odorless air.

Then, an ammonia gas was injected into the Tedlar bag and the concentration of ammonia in the container after 60 minutes and 120 minutes was measured, respectively, using a gas detecting tube (manufactured by Gestec Service, Inc.). A hydrogen sulfide gas was injected in the same manner as in the above and the concentration of hydrogen sulfide was measured.

A deodorization rate was calculated in accordance with Equation described in Example 1. The Results Are Shown in Table 3 in Addition, an Initial Concentration Of ammonia was 40 ppm and an initial concentration of hydrogen sulfide was 10 ppm.

### Comparative Example 3

### Deodorization of Effect of Lauryl Methacrylate on Ammonia and Hydrogen Sulfide Odors

Lauryl methacrylate was used as a sample, the same adjustment as in Example 3 was performed and the concentration of ammonia and hydrogen sulfide was measured. The results are shown in Table 3.

TABLE 3
Results of deodorization test on ammonia and hydrogen sulfide odors

| | | Ammonia | | Hydrogen sulfide | |
|---|---|---|---|---|---|
| | | Deodorization | Deodorization | Deodorization | Deodorization |
| | | rate after 60 min | rate after 120 min | rate after 60 min | rate after 120 min |
| | Sample name | (%) | (%) | (%) | (%) |
| Example 3 | Mixture of | 84.0 | 92.0 | 92.0 | 100.0 |
| | hydrogenated | | | | |
| | dimer acid, | | | | |
| | zinc | | | | |
| | ricinoleate and | | | | |
| | alcohol-based | | | | |
| | solvent | | | | |
| Comparative | Lauryl | 12.0 | 18.0 | 00.0 | 00.0 |
| Example 3 | methacrylate | | | | |

As can be seen from Table 3, the mixture of hydrogenated dimer acid and zinc ricinoleate exhibited superior deodorization effects on ammonia odor and hydrogen sulfide odor.

### Example 4-1

### Deodorization Effects of Mixture of Blended-Fragrance and Hydrogenated Dimer Acid on Ammonia Odor

To a filter paper having φ55 mm, 1.0 g of 20% by weight of an isoparaffin solution of a sample in which 50% by weight of lemon verbena-blended fragrance and 50% by weight of hydrogenated dimer acid are mixed and adjusted, was added dropwise, followed by addition to a 5 L Tedlar bag, sealing and injection of odorless air.

Then, an ammonia gas was injected into the Tedlar bag and the concentration of ammonia in the container after 60 minutes and 120 minutes was measured, respectively, using a gas detecting tube (manufactured by Gestec Service, Inc.). A deodorization rate was calculated in accordance with Equation described in Example 1. The results are shown in Table 4. In addition, an initial concentration of ammonia was 40 ppm.

### Example 4-2

### Deodorization of Effect of Hydrogenated Dimer Acid on Ammonia Odor

Hydrogenated dimer acid was used as a sample, the same adjustment as in Example 4-1 was performed and the concentration of ammonia was measured. The results are shown in Table 4.

### Comparative Example 4

### Deodorization Effects of Lauryl Methacrylate on Ammonia Odor

A sample in which 50% by weight of lemon verbena-blended fragrance and 50% by weight of lauryl methacrylate are mixed and adjusted was used, the same adjustment as in Example 4 was performed and the concentration of ammonia was measured. The results are shown in Table 4.

TABLE 4
Results of deodorization test on ammonia odor (initial concentration 40 ppm)

| | | Deodorization | Deodorization |
|---|---|---|---|
| | | rate after 60 min | rate after 120 min |
| | Sample name | (%) | (%) |
| Example | Mixture of hydrogenated dimer acid | 75.0 | 94.0 |
| 4-1 | and lemon verbena-blended fragrance | | |
| Example | Hydrogenated dimer acid | 75.0 | 94.0 |
| 4-2 | | | |
| Comparative | Mixture of lauryl methacrylate and | 13.0 | 18.0 |
| Example 4 | lemon verbena-blended fragrance | | |

As can be seen from Table 4, the mixture of hydrogenated dimer acid and lemon verbena-blended fragrance exhibited superior deodorization effects on ammonia odor, as compared to the mixture of lauryl methacrylate and lemon verbena-blended fragrance.

While the invention has been described in detail and with reference to specific embodiments thereof, it will be apparent to one skilled in the art that various changes and modifications can be made therein without departing from the spirit and scope thereof.

This application is based on Japanese Patent Application No. 2011-048207 filed on Mar. 4, 2011, the entire subject matter of which is incorporated herein by reference. In addition, the subject matters of all documents cited in the specification are also incorporated here by reference.

### INDUSTRIAL APPLICABILITY

The oil-soluble deodorant composition of the present invention contains at least one of dimer acids and trimer acids as an active ingredient, and thus the composition exhibits excellent deodorization effects on odors of ammonia, amines such as trimethylamine and the like, as compared to conventional oil-soluble deodorant compositions.

In addition, the dimer acids contained in the oil-soluble deodorant composition of the present invention have a skeleton of dicarboxylic acid and the trimer acids therein have a skeleton of tricarboxylic acid. The dimer and trimer acids are readily dissolved in oil-soluble solvent such as hydrocarbon and are thus considerably useful.

Furthermore, dimer acids and trimer acids are liquid substances that are flowable in spite of their large molecular weights and the oil-soluble deodorant composition of the present invention is excellent in terms of handling.

In addition, since the oil-soluble deodorant composition of the present invention is free of an irritant odor or the like, it is highly safe and may be added to various products, thus being considerably useful.

1. An oil-soluble deodorant composition comprising at least one of a dimer acids and a trimer acids as an active ingredient.

2. The oil-soluble deodorant composition according to claim 1, wherein the dimer acids is at least one of a dimer acid and a hydrogenated dimer acid.

3. The oil-soluble deodorant composition according to claim 1, wherein the trimer acids is at least one of a trimer acid and a hydrogenated trimer acid.

4. The oil-soluble deodorant composition according to claim 1, further comprising at least one of a fatty acid metal salt and an organic acid metal salt.

5. The oil-soluble deodorant composition according to claim 4, wherein the metal of the at least one of the fatty acid metal salt and the organic acid metal salt is one or more selected from the group consisting of sodium, potassium, calcium, copper, iron, zinc and magnesium.

6. The oil-soluble deodorant composition according to claim 4, wherein the fatty acid metal salt is zinc ricinoleate.

7. The oil-soluble deodorant composition according to claim 1, wherein a total content of the dimer acids and trimer acids is 0.1% by weight or more.

8. A method for deodorizing ammonia odor or amine odor using at least one of a dimer acids and a trimer acids.

9. A method for deodorizing ammonia odor or amine odor, comprising applying at least one of a dimer acids and a trimer acids to a subject to be deodorized, or applying at least one of a dimer acids and a trimer acids near a subject to be deodorized.

10. The oil-soluble deodorant composition according to claim 2, further comprising at least one of a fatty acid metal salt and an organic acid metal salt.

11. The oil-soluble deodorant composition according to claim 3, further comprising at least one of a fatty acid metal salt and an organic acid metal salt.

12. The oil-soluble deodorant composition according to claim 10, wherein the metal of the at least one of the fatty acid metal salt and the organic acid metal salt is one or more selected from the group consisting of sodium, potassium, calcium, copper, iron, zinc and magnesium.

13. The oil-soluble deodorant composition according to claim 11, wherein the metal of the at least one of the fatty acid metal salt and the organic acid metal salt is one or more selected from the group consisting of sodium, potassium, calcium, copper, iron, zinc and magnesium.

14. The oil-soluble deodorant composition according to claim 10, wherein the fatty acid metal salt is zinc ricinoleate.

15. The oil-soluble deodorant composition according to claim 11, wherein the fatty acid metal salt is zinc ricinoleate.

16. The oil-soluble deodorant composition according to claim 2, wherein a total content of the dimer acids and trimer acids is 0.1% by weight or more.

17. The oil-soluble deodorant composition according to claim 3, wherein a total content of the dimer acids and trimer acids is 0.1% by weight or more.

18. The oil-soluble deodorant composition according to claim 4, wherein a total content of the dimer acids and trimer acids is 0.1% by weight or more.

19. The oil-soluble deodorant composition according to claim 10, wherein a total content of the dimer acids and trimer acids is 0.1% by weight or more.

20. The oil-soluble deodorant composition according to claim 11, wherein a total content of the dimer acids and trimer acids is 0.1% by weight or more.
